(19) United States
(12) Patent Application Publication
(10) Pub. No.: US 2007/0258931 A1
(43) Pub. Date: Nov. 8, 2007

(54) Cosmetic compositions comprising an oxyethylene sorbitol ester and a cyclodextrain, processes and uses

(76) Inventor: Rainer Muller, Leopoldshafen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/790,725

(22) Filed: Apr. 27, 2007

Related U.S. Application Data
(60) Provisional application No. 60/799,031, filed on May 10, 2006.

(30) Foreign Application Priority Data
Apr. 27, 2006 (FR) 06/03808

(51) Int. Cl. A61K 8/73; A61K 8/37

(52) U.S. Cl. 424/70.7; 424/70.13; 424/70.31

(57) ABSTRACT

The present disclosure relates to a cosmetic composition comprising, in a physiologically acceptable aqueous medium, at least one oxyethylenated C₈ to C₁₄ fatty acid ester of sorbitan comprising from 2 to 10 oxyethylene units, and at least one cyclodextrin or derivative thereof. The present disclosure also relates to a process for cosmetic treatment, for example for washing and caring for keratin fibers, such as the hair, using said composition, and also to the uses of these cosmetic compositions, for example for treating, such as washing, keratin fibers, such as the hair.

This application claims benefit of U.S. Provisional Application No. 60/799,031, filed May 10, 2006, the contents of which are incorporated herein by reference. This application also claims benefit of priority under 35 U.S.C. § 119 to French Patent Application No. FR 06/03808, filed Apr. 27, 2006, the contents of which are also incorporated herein by reference.

The present disclosure relates to the field of cosmetic compositions, for example, washing cosmetic compositions such as shampoos comprising pearlescent agents.

Many compositions for washing keratin materials have been described in the prior art. In addition to their washing properties and their cosmetic qualities, washing compositions, such as shampoos, should ideally not be aggressive to the skin or the eyes. This quality is appreciated, for example, in shampoos for children.

Cosmetic products which have an iridescent, shimmering or metallized appearance or effect are also widely favored by consumers on account of their aesthetic appeal since they can give the product an appearance of richness. The agents which provide this effect can be pearling agents or pearlescent agents generally comprising crystals which remain dispersed in the compositions and which reflect light.

Long-chain ester derivatives are widely used for pearling compositions, for example, cosmetic compositions. However, these derivatives can present crystallization problems which can lead to a change in the viscosity of the compositions over time.

Long-chain ether or thioether derivatives such as those described in European application EP 457 688 and PCT application WO 98/03155 are also known. However, these agents can opacify the compositions without giving them any, or giving them insufficient, pearling effect.

It has also been observed that these pearlescent agents, due to their low density, often have the drawback of rising to the surface of the shampoo and of forming there a layer that consumers find unattractive.

In addition, these fatty-chain compounds may have the drawback, in certain cases, of giving the hair a laden feel and of producing a head of hair that lacks lightness and volume.

All these pearlescent agents are water-insoluble compounds and have a melting point above 50° C. In order to produce pearlescent compounds, it may be necessary to heat the compositions above the melting point of the pearlescent compound, then to cool, and subsequently to add the other compounds of the composition. In order to decrease the consumption of energy and to reduce the production time, it is desirable to prepare the compositions under cold conditions.

PCT Application WO 03/088934 describes the use of a cyclodextrin as a pearlescent agent which can be used under cold conditions. However, the compositions containing a cyclodextrin may not be sufficiently stable.

Thus, there is a need in the art for obtaining pearlescent cosmetic compositions, for example, washing compositions such as shampoos, which are stable over time and which are not aggressive to the skin and the eyes.

For the purpose of the present disclosure, the term “stable” is intended to mean that the visual appearance and the feel of these compositions does not substantially change over time under normal storage conditions, for example during the 12, 24, and in at least one embodiment, 30 months following their production. In at least one embodiment, this means that no phase separation of the compositions according to the present disclosure is observed, the pearlescent agents contained in these compositions do not rise to the surface.

For the purpose of the present disclosure, the term “nonaggressive” is intended to mean that the composition does not cause substantially any stinging of the eyes when it reaches the ocular sphere, and more generally does not cause any reactions such as red blotches, itching or stinging, for example, in users with sensitive skin.

The term “keratin materials” is understood herein to mean the hair, the eyelashes, the eyebrows, the skin, the nails, the mucous membranes or the scalp, for example the hair, and the term “keratin fibers” is understood herein to mean the hair, the eyelashes and the eyebrows.

As defined herein, the term “pearling agent” or “pearlescent agent” is intended to mean a product that produces a pearlescent, iridescent, shimmering or metallized appearance or effect.

For the purpose of the present disclosure, the term “washing composition” is intended to mean a composition comprising at least 4% by weight of at least one surfactant, for example, from 4% to 50% by weight of at least one surfactant, relative to the total weight of the composition.

The present inventor has discovered that it is possible to formulate stable and nonaggressive cosmetic compositions for the treatment of keratin materials, for example, shampoos, which have a pearlescent appearance while at the same time having the desired washing, aesthetic and cosmetic properties, by using at least one oxyethylenated C₈ to C₁₄ fatty acid ester of sorbitan comprising from 2 to 10 oxyethylene units (OE), and at least one cyclodextrin or derivative thereof.

The present disclosure therefore relates to a cosmetic composition, for example, a washing composition such as a shampoo, comprising, in a physiologically acceptable aqueous medium, at least one oxyethylenated C₈ to C₁₄ fatty acid ester of sorbitan comprising from 2 to 10 oxyethylene units, and at least one cyclodextrin or derivative thereof.

The present disclosure also relates to a process for cosmetic treatment, for example, for washing keratin fibers such as the hair, using the disclosed composition, and also to the uses of these cosmetic compositions, for example, for treating, such as washing, keratin fibers such as the hair.

The present disclosure is also directed towards the uses of the composition according to the present disclosure, for example, as a composition for the cosmetic treatment or care of keratin materials and/or as a shampoo or a composition to be applied before or after a shampoo.

The compositions according to the present disclosure may exhibit very good homogeneity and good stability of the pearling, for application on keratin materials. In at least one embodiment, there is no phase separation.

Also, in at least one embodiment, no uncontrolled graining-out or thickening of the composition over time takes place. The end result of this is that the compositions have a non-runny, fondant texture. The lather in such a case is easy to rinse out.

Other possible characteristics, aspects, subjects and advantages of the present disclosure will emerge even more clearly on reading the description and the examples which follow.

The composition which is the subject of the present disclosure comprises a nonionic surfactant comprising at least one oxyethylenated C₈ to C₁₄ fatty acid ester of sorbitan comprising from 2 to 10 oxyethylene units.

In at least one embodiment, the composition according to the present disclosure comprises at least one oxyethylenated C₁₂ fatty acid ester of sorbitan comprising from 2 to 10 oxyethylene units, for example, 4 oxyethylene units.

In at least one further embodiment, the composition according to the present disclosure comprises oxyethylene sorbitan monolaurate comprising 4 oxyethylene units (OE). This compound is also known as polysorbate 21. It is, inter alia, sold under the name TWEEN 21 by the company Uniqema.

The composition according to the present disclosure, in at least one embodiment, comprises at least 0.5% by weight of the at least one oxyethylenated C₈ to C₁₄ fatty acid ester of sorbitan comprising from 2 to 10 oxyethylene units, relative to the total weight of the composition. For example, it comprises from 0.5% to 10% by weight of the at least one oxyethylenated C₈ to C₁₄ fatty acid ester of sorbitan comprising from 2 to 10 oxyethylene units, such as from 2% to 9% by weight, and in at least one embodiment, from 4% to 8% by weight, relative to the total weight of the composition.

The at least one oxyethylenated C₈ to C₁₄ fatty acid ester of sorbitan comprising from 2 to 10 oxyethylene units may be used alone, or as a mixture with at least one other oxyethylenated derivative of sorbitan. For example, in at least one embodiment, also employed in the compositions according to the disclosure is at least one oxyethylenated C₈ to C₂₄ fatty acid ester of sorbitan comprising from 15 to 50 oxyethylene units.

These compositions can present, in at least one embodiment, an improved pearlescent aspect, visible to the naked eye and persistent over time, and desired aesthetic properties. For example, the compositions may be less yellowed.

In at least one embodiment, the at least one other oxyethylenated derivative of sorbitan is an oxyethylenated C₈ to C₂₄ fatty acid monoester of sorbitan comprising 20 oxyethylene units.

In at least one further embodiment, the at least one other oxyethylenated derivative of sorbitan is oxyethylenated sorbitan monolaurate comprising 20 oxyethylene units.

The composition according to the disclosure, in at least one embodiment, comprises from 0.1% to 10% by weight of oxyethylenated C₈ to C24 fatty acid monoester of sorbitan comprising from 15 to 50 oxyethylene units, for example, from 0.5% to 5% by weight, relative to the total weight of the composition.

The at least one cyclodextrin or derivative thereof that can be used as pearlescent agent in the compositions according to the present disclosure is chosen from, for example, oligosaccharides of formula: 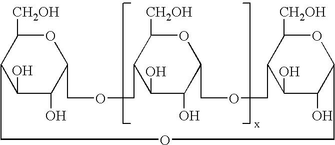
wherein x is chosen from 4 (corresponding to α-cyclodextrin), 5 (corresponding to β-cyclodextrin) and 6 (corresponding to γ-cyclodextrin).

In at least one embodiment, the at least one cyclodextrin or derivative thereof is chosen from β-cyclodextrin and γ-cyclodextrin, for example, β-cyclodextrin.

A β-cyclodextrin sold by the company WACKER under the name CAVAMAX W7 PHARMA and a γ-cyclodextrin sold by the company WACKER under the name CAVAMAX W8 can, for example, be used.

The at least one cyclodextrin or derivative thereof can be chosen from, for example, methylcyclodextrins such as the methyl-β-cyclodextrin sold by the company WACKER under the name CAVASOL W7.

According to the present disclosure, the at least one cyclodextrin or derivative thereof is present in a range from 1% to 15% by weight, such as from 1% to 10% by weight, and for example, from 1.5% to 5% by weight, relative to the total weight of the composition.

The composition according to the present disclosure can contain at least one additional surfactant. The at least one cyclodextrin or derivative thereof and all the surfactants (including the oxyethylenated sorbitol ester(s)) are present in an effective concentration for giving the composition a pearling effect and/or for forming a complex, insoluble in the composition, between the at least one cyclodextrin or derivative thereof and at least one surfactant.

According to at least one embodiment, the at least one cyclodextrin or derivative thereof is introduced into the composition in noncomplexed form or optionally in a form complexed with at least one surfactant, i.e. when a complex is formed, it has not been formed with a compound other than a surfactant.

In the composition, the at least one cyclodextrin or derivative thereof is, in at least one embodiment, complexed with at least one surfactant.

The weight ratio of all the surfactants (including the oxyethylenated sorbitol ester(s)) to the at least one cyclodextrin or derivative thereof can range from 0.01 to 300, such as from 0.1 to 100, and for example from 0.3 to 25.

As additional surfactants that can be used in the compositions of the disclosure, mention may be made of the surfactants conventionally used in this field, such as, but not limited to, anionic surfactants, additional nonionic surfactants other than the oxyethylenated sorbitan derivatives, amphoteric or zwitterionic surfactants, or cationic surfactants.

The anionic surfactants that can be used in the compositions according to the present disclosure are, for example, chosen from salts, for example alkali metal salts such as sodium salts, ammonium salts, amine salts, amino alcohol salts or alkaline-earth metal, for example magnesium, salts, of the following types: alkyl sulphates, alkyl ether sulphates, alkylamido ether sulphates, alkylarylpolyether sulphates, monoglyceride sulphates, alkyl sulphonates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphoacetates, acyl sarcosinates and acyl glutamates, wherein the alkyl and acyl groups of all these compounds have from 6 to 24 carbon atoms and the aryl group, in at least one embodiment, is chosen from a phenyl and a benzyl group.

It is also possible to use monoesters of C₆-C₂₄ alkyl and of polyglycoside-dicarboxylic acids, such as alkyl glucoside citrates, polyalkyl glycoside tartrates and polyalkyl glycoside sulphosuccinates, alkyl sulphosuccinamates, acyl isothionates and N-acyltaurates, wherein the alkyl and acyl groups of all these compounds have from 12 to 20 carbon atoms.

Another group of anionic surfactants that can be used in the compositions of the present disclosure is that of acyl lactylates wherein the acyl group have from 8 to 20 carbon atoms.

In addition, mention may also be made of alkyl-D-galactosiduronic acids and salts thereof, and also polyoxyalkylenated (C₆-C₂₄ alkyl) ether-carboxylic acids, polyoxyalkylenated (C₆-C₂₄ alkyl)(C₆-C₂₄ aryl) ether-carboxylic acids, polyoxyalkylenated (C₆-C₂₄ alkyl)amidoether-carboxylic acids and salts thereof, for example those comprising from 2 to 50 ethylene oxide units, and mixtures thereof.

Alkyl sulphates, alkyl ether sulphates and mixtures thereof, for example in the form of alkali metal, alkaline-earth metal, ammonium, amine or amino alcohol salts, are used in at least one embodiment.

When present, the amount of the anionic surfactant(s) ranges,for example, from 0.5% to 50% by weight, such as from 4% to 20% by weight, relative to the total weight of the composition.

Non-limiting examples of additional nonionic surfactants that can be used in the compositions of the present disclosure are described, for example, in “Handbook of Surfactants” by M. R. Porter, published by Blackie & Son (Glasgow and London), 1991, pp. 116-178. The additional nonionic surfactants are, for example, chosen from, but not limited to alcohols, alpha-diols, (C₁-C₂₀)alkylphenols and fatty acids which are polyoxyethylated, polypropoxylated or polyglycerylated, having a fatty chain containing, for example, from 8 to 18 carbon atoms, it being possible for the number of ethylene oxide or propylene oxide groups to range, for example, from 2 to 50 and it being possible for the number of glycerol groups to range, for example, from 2 to 30.

Mention may also be made in a non-limiting manner of condensates of ethylene oxide and of propylene oxide with fatty alcohols; polyethoxylated fatty amides, for example having from 2 to 30 ethylene oxide units, polyglycerylated fatty amides comprising, on average, from 1 to 5 glycerol groups, such as from 1.5 to 4, fatty acid esters of sucrose, fatty acid esters of polyethylene glycol, (C₆-C₂₄ alkyl)polyglycosides, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

When present, the amount of the additional nonionic surfactant(s) ranges, for example, from 0.01% to 10% by weight, such as from 0.05% to 5% by weight, relative to the total weight of the composition.

The compositions of the disclosure can also comprise at least one amphoteric or zwitterionic surfactant.

The at least one amphoteric or zwitterionic surfactant that may be used in the compositions according to the disclosure can, for example, be derivatives of secondary or tertiary aliphatic amines, wherein the aliphatic group is chosen from linear and branched chains having from 8 to 22 carbon atoms and having at least one anionic group such as, for example, a carboxylate, sulphonate, sulphate, phosphate or phosphonate group. Non-limiting mention may also be made of (C₈-C₂₀)alkylbetaines, sulphobetaines, (C₈-C₂₀ alkyl)amido(C₆-C₈ alkyl)betaines and (C₈-C₂₀ alkyl)amido(C₆-C₈ alkyl)sulphobetaines.

Among the amine derivatives, mention may be made of the products sold under the name Miranol®, as described in U.S. Pat. Nos. 2,528,378 and 2,781,354 and classified in the CTFA dictionary, 3^{rd} edition, 1982, under the names amphocarboxyglycinate and amphocarboxypropionate having the respective structures (I) and (II):
Rₐ—CONHCH₂CH₂—N(R_{b})(R_{c})(CH₂COO⁻) (I)
wherein:
Rₐ is chosen from an alkyl group derived from an acid Rₐ—COOH present in hydrolyzed coconut oil, and from heptyl, nonyl and undecyl groups,
R_{b} is a beta-hydroxyethyl group, and
R_{c} is a carboxymethyl group; and
   Rₐ′—CONHCH₂CH₂—N(B)(B′) (II)
   wherein:
B is —CH₂CH₂OX′,
B′ is —(CH₂)_{z}—Y′, wherein z=1−2,
X′ is chosen from the group —CH₂CH₂—COOH and a hydrogen atom,
Y′ is chosen from the group —COOH and the group —CH₂—CHOH—SO₃H,
R′ₐ is chosen from alkyl groups of acids R′ₐ—COOH present in coconut oil and in hydrolyzed linseed oil, alkyl groups, for example a C₁₇ alkyl group and its iso form, or an unsaturated C₁₇ group.

These compounds are classified in the CTFA dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M concentrate.

Among the amphoteric or zwitterionic surfactants mentioned above, use is, in at least one embodiment, made of (C₈-C₂₀ alkyl)betaines, (C₈-C₂₀ alkyl)amido(C₆-C₈ alkyl)betaines and mixtures thereof.

When present, the amount of the amphoteric or zwitterionic surfactant(s) ranges, for example, from 0.1% to 10% by weight, such as from 0.5% to 8% by weight, relative to the total weight of the composition.

The composition according to the present disclosure, in at least one embodiment, has a total anionic, nonionic, amphoteric and zwitterionic surfactant content ranging from 4% to 50% by weight, such as from 4% to 20% by weight, relative to the total weight of the composition.

The composition of the disclosure, in at least one embodiment, has at least 4% of at least one anionic surfactant.

The compositions according to the present disclosure can also comprise at least one cationic surfactant.

By way of examples of cationic surfactants, mention may be made of the salts of primary, secondary or tertiary amines, which are optionally polyoxyalkylenated; quaternary ammonium salts, such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides that are cationic in nature.

When the cationic surfactants are present, their amount ranges, for example, from 0.01% to 10% by weight, such as from 0.05% to 5% by weight, and for example from 0.3% to 3% by weight, relative to the total weight of the cosmetic composition.

As a non-limiting example, the composition according to the present disclosure comprises at least one anionic surfactant and at least one amphoteric or zwitterionic surfactant.

The compositions according to the present disclosure can also comprise at least one cationic polymer.

The composition according to the present disclosure can comprise an amount, for example, ranging from 0.01 to 10% by weight of the at least one cationic polymer, such as from 0.1 to 5% by weight, relative to the total weight of the composition.

The compositions according to the present disclosure can also comprise at least one mineral or organic salt such as sodium chloride. The at least one mineral or organic salt may be present in an amount ranging from 0.1 to 10% by weight, for example, from 0.5 to 5% by weight, relative to the total weight of the composition.

In at least one embodiment, the at least one mineral or organic salt is added to a composition according to the present disclosure comprising at least one oxyethylenated C₈ to C₁₄ fatty acid ester of sorbitan comprising from 2 to 10 oxyethylene units, at least one oxyethylenated C₈ to C₂₄ fatty acid ester of sorbitan comprising from 15 to 50 oxyethylene units, and at least one cyclodextrin or derivative thereof.

According to at least one embodiment, the oxyethylenated C₈ to C₁₄ fatty acid ester of sorbitan comprising from 2 to 10 oxyethylene units is an oxyethylenated C₁₂ fatty acid ester of sorbitan comprising from 2 to 10 oxyethylene units, for example, 4 oxyethylene units. In at least one further embodiment, the ester is an oxyethylene sorbitan monolaurate comprising 4 oxyethylene units.

According to at least one embodiment, the oxyethylenated C₈ to C₂₄ fatty acid ester of sorbitan comprising from 15 to 50 oxyethylene units is an oxyethylenated C₈ to C₂₄ fatty acid ester of sorbitan comprising 20 oxyethylene units. In at least one emodiment, the ester is an oxyethylene sorbitan monolaurate comprising 20 oxyethylene units.

The compositions according to the disclosure, in at least one embodiment, present a dynamic viscosity ranging from 2 to 8 Pa·s. and, for example from 2.5 to 5.5 Pa·s., at room temperature (25° C.). These measurements of viscosity can be made, in at least one embodiment, via a Rheomat R180 type rheometer, with half-minute intervals between each measurement and with a shear rate of 200 mN.m.

The aqueous medium consists of water or a mixture of water and at least one cosmetically acceptable solvent chosen from C₁-C₄ lower alcohols, such as ethanol, isopropanol, tert-butanol or n-butanol; polyols such as glycerol, hexylene glycol, propylene glycol or polyethylene glycols, and mixtures thereof.

The pH of the compositions according to the disclosure can be less than 8.5, for example, ranging from 4 to 7.

The composition according to the disclosure can also comprise at least one conventional additive well known in the art, such as agents for preventing hair loss, oxidizing agents, ceramides and pseudoceramides, vitamins and provitamins, including panthenol, plant, animal, mineral or synthetic oils, waxes, sunscreens, colored or colorless, inorganic or organic pigments, dyes, sequestering agents, plasticizers, solubilizing agents, acidifying agents, basifying agents, inorganic or organic thickeners, antioxidants, hydroxy acids, fragrances and preserving agents.

Those skilled in the art will take care to choose the optional additives and the amounts thereof in such a way that they do not harm the properties of the compositions of the present disclosure.

The at least one conventional additive can be present in the composition according to the present disclosure in an amount ranging from 0% to 20% by weight relative to the total weight of the composition.

The composition according to the present disclosure can be used, for example, as a composition for the cosmetic treatment or care of keratin materials.

As used herein, the term “keratin materials” is understood to mean the hair, the eyelashes, the eyebrows, the skin, the nails, the mucous membranes or the scalp, for example, the hair.

The composition according to the present disclosure can be used, for example, as a shampoo or a composition to be applied before or after a shampoo.

The present disclosure also relates to a cosmetic treatment process, which comprises the application to the hair of an effective amount of a cosmetic composition as described above.

According to at least one embodiment, such a process comprises applying to the hair an effective amount of the cosmetic composition, and optionally rinsing it off after an optional waiting time.

When the composition according to the disclosure is applied in the form of a lotion or of a cream before or after shampooing, it is optionally left on the hair for approximately ½ minutes to 5 minutes, optionally followed by rinsing with water.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The following examples are given by way of illustration of the present disclosure and cannot limit the scope thereof.

### EXAMPLES

In the following example, the amounts are indicated as percentage by weight of active material (AM) relative to the total weight of the composition, unless otherwise indicated.

### Example 1

The following shampoo composition A was prepared:

| COMPOSITION | A |
|---|---|
| 1,3-dimethylol-5,5-dimethylhydantoin as an aqueous | 0.14 |
| solution (55% by weight) | |
| Sodium salt of methyl p-hydroxybenzoate | 0.2 |
| Hydroxypropylguar-trimethylammonium chloride (Jaguar | 0.1 |
| C13S sold by Rhodia) | |
| Carboxyvinylpolymer (Carbopol 981 sold by Noveon) | 0.2 |
| Beta-cyclodextrin (cyclomatoheptaose) (Cavamax W7 | 1.72 |
| Pharma sold by Wacker) | |
| Sodium chloride | 0.1 |
| Polydimethylsiloxane | 2.7 |
| Oxyethylenated sorbitan monolaurate (4 OE) (Tween 21 | 6 |
| sold by Uniqema) | |
| Cocoylamidopropylbetaine as an aqueous solution (47% | 2.43 |
| by weight) | |
| Sodium lauryl ether sulphate (2.2 OE) as an aqueous | 15.5 |
| solution (70% by weight) | |
| Fragrance | 0.5 |
| Pure sodium hydroxide/citric acid, 1H₂O | pH 6.5-7.5 |
| q.s. | |
| Deionized water q.s. | 100 |

The composition A was found to give an excellent tolerance with respect to the scalp. For example, very few discomforting reactions were observed. In addition, this composition displayed excellent ocular tolerance.

Finally, this composition was stable and displayed good qualities of use, and notable cosmetic properties.

### Examples 2 and 3

The following shampoo compositions B and C were prepared:

| COMPOSITIONS | B | C |
|---|---|---|
| Cocoylamidopropylbetaine (Tego Betaine F50 | 2.4 | 2.4 |
| sold by Goldschmidt Degussa) | | |
| Dimethicone (Dow Corning 200 Fluid 500 000 | 1.5 | 1.5 |
| cSt sold by Dow Corning) | | |
| Polyquaternium-10 (Ucare Polymer JR 400 LT | 0.4 | 0.4 |
| sold by Amerchol-Dow Chemical) | | |
| Sodium laureth sulfate (Texapon AOS 225 UP | 15.4 | 15.4 |
| sold by Cognis) | | |
| Beta-cyclodextrin (Cavamax W7 Pharma sold by | 1.7 | 1.7 |
| Wacker) | | |
| Polysorbate 21 (Tween 21 sold by Uniqema) | 6 | 2 |
| Polysorbate 20 (Tween 20 sold by Uniqema) | — | 4 |
| Carbomer (Carbopol 980 sold by Noveon) | 0.2 | 0.2 |
| Sodium chloride⁽*⁾ | q.s. | q.s. |
| Preservatives | q.s. | q.s. |
| Pure sodium hydroxide/citric acid, 1H₂O | pH 5-5.6 | pH 5-5.6 |
| q.s. | | |
| Deionized water q.s. | 100 | 100 |

⁽*⁾added in sufficient quantity to get a viscosity ranging from 3 to 5.5 Pa.s. at room temperature (about 25° C.), said viscosity being measured by a Rheomat R180 type rheometer, with half-minute intervals between each measurement and with a shear rate of 200 mN · m.

The compositions B and C presented a pearlescent aspect, visible to the naked eye.

What is claimed is:

1. A composition for caring for keratin materials, comprising, in an aqueous medium:
at least one cyclodextrin or derivative thereof, and
at least one oxyethylenated C₈ to C₁₄ fatty acid ester of sorbitan comprising from 2 to 10 oxyethylene units.

2. The composition according to claim 1, wherein the at least one oxyethylenated C₈ to C₁₄ fatty acid ester of sorbitan is an oxyethylenated C₁₂ fatty acid ester of sorbitan comprising from 2 to 10 oxyethylene units.

3. The composition according to claim 2, wherein the at least one oxyethylenated C₈ to C₁₄ fatty acid ester of sorbitan is oxyethylene sorbitan monolaurate comprising 4 oxyethylene units.

4. The composition according to claim 1, wherein the at least one oxyethylenated C₈ to C₁₄ fatty acid ester of sorbitan comprising from 2 to 10 oxyethylene units is present in an amount of at least 0.5% by weight, relative to the total weight of the composition.

5. The composition according to claim 4, wherein the at least one oxyethylenated C₈ to C₁₄ fatty acid ester of sorbitan comprising from 2 to 10 oxyethylene units is present in an amount of from 2% to 9% by weight, relative to the total weight of the composition.

6. The composition according to claim 1, wherein the at least one cyclodextrin is chosen from α-cyclodextrin, β-cyclodextrin, γ-cyclodextrin and derivatives thereof.

7. The composition according to claim 6, wherein the at least one cyclodextrin is chosen from β-cyclodextrin and γ-cyclodextrin.

8. The composition according to claim 7, wherein the at least one cyclodextrin is β-cyclodextrin.

9. The composition according to claim 1, wherein the at least one cyclodextrin is present in an amount ranging from 1% to 15% by weight, relative to the total weight of the composition.

10. The composition according to claim 9, wherein the at least one cyclodextrin is present in an amount ranging from 1.5% to 5%, relative to the total weight of the composition.

11. The composition according to claim 1, wherein the composition is a washing composition.

12. The composition according to claim 11, wherein said composition comprises at least one surfactant present in a total surfactant content of at least 4% by weight, relative to the total weight of the composition.

13. The composition according to claim 12, wherein the total surfactant content ranges from 4% to 50% by weight, relative to the total weight of the composition.

14. The composition according to claim 1, wherein the composition further comprises at least one oxyethylenated C₈ to C₂₄ fatty acid monoester of sorbitan comprising from 15 to 50 oxyethylene units.

15. The composition according to claim 14, wherein the at least one oxyethylenated C₈ to C₂₄ fatty acid monoester of sorbitan is an oxyethylenated C₈ to C₂₄ fatty acid monoester of sorbitan comprising 20 oxyethylene units.

16. The composition according to claim 15, wherein the at least one oxyethylenated C₈ to C₂₄ fatty acid monoester of sorbitan is oxyethylene sorbitan monolaurate comprising 20 oxyethylene units.

17. The composition according to claim 14, wherein the at least one oxyethylenated C₈ to C₂₄ fatty acid monoester of sorbitan comprising from 15 to 50 oxyethylene units is present in an amount ranging from 0.1% to 10% by weight, relative to the total weight of the composition.

18. The composition according to claim 17, wherein the at least one oxyethylenated C₈ to C₂₄ fatty acid monoester of sorbitan comprising from 15 to 50 oxyethylene units is present in an amount ranging from 0.5% to 5% by weight, relative to the total weight of the composition.

19. The composition according to claim 12, wherein the composition further comprises at least one additional surfactant chosen from anionic, nonionic and amphoteric or zwitterionic surfactants.

20. The composition according to claim 19, wherein the composition comprises at least one anionic surfactant and at least one amphoteric or zwitterionic surfactant.

21. The composition according to claim 19; wherein the at least one anionic surfactant is chosen from alkyl sulphates, alkyl ether sulphates, and mixtures thereof.

22. The composition according to claim 19, wherein the at least one anionic surfactant is present in an amount ranging from 0.5% to 50% by weight, relative to the total weight of the composition.

23. The composition according to claim 22, wherein the at least one anionic surfactant is present in an amount ranging from 4% to 20% by weight, relative to the total weight of the composition.

24. The composition according to claim 19, wherein the at least one amphoteric or zwitterionic surfactant is chosen from (C₈-C₂₀ alkyl)betaines, (C₈-C₂₀ alkyl)amido(C₆-C₈ alkyl)betaines, and mixtures thereof.

25. The composition according to claim 19, wherein the composition further comprises at least one cationic surfactant.

26. The composition according to claim 1, wherein the composition further comprises at least one cationic polymer.

27. The composition according to claim 26, wherein the at least one cationic polymer is present in an amount ranging from 0.01% to 10% by weight, relative to the total weight of the composition.

28. The composition according to claim 27, wherein the at least one cationic polymer is present in an amount ranging from 0.1% to 5% by weight, relative to the total weight of the composition.

29. The composition according to claim 1, wherein the aqueous medium consists of water or a mixture of water and at least one cosmetically acceptable solvent.

30. The composition according to claim 29, wherein the at least one cosmetically acceptable solvent is chosen from C₁-C₄ lower alcohols, and polyols.

31. The composition according to claim 1, wherein the composition further comprises at least one conventional additive chosen from agents for preventing hair loss, oxidizing agents, ceramides and pseudoceramides, vitamins and provitamins, plant, animal, mineral and synthetic oils, waxes, sunscreens, colored and colorless, inorganic and organic pigments, dyes, sequestering agents, plasticizers, solubilizing agents, acidifying agents, basifying agents, inorganic and organic thickeners, antioxidants, hydroxy acids, fragrances and preserving agents.

32. The composition according to claim 31, wherein the at least one conventional additive is chosen from panthenol.

33. A process for the cosmetic treatment or care of keratin materials, said process comprising applying to said keratin materials an effective amount of a composition comprising, in an aqueous medium: at least one cyclodextrin or derivative thereof, and at least one oxyethylenated C₈ to C₁₄ fatty acid ester of sorbitan comprising from 2 to 10 oxyethylene units.

34. The process according to claim 32, wherein said composition is in the form of a shampoo or a composition to be applied before or after a shampoo.
